(19) United States
(12) Patent Application Publication
(10) Pub. No.: US 2026/0061157 A1
(43) Pub. Date: Mar. 5, 2026

(54) THERAPEUTIC VEST

(71) Applicant: Samuel J. Forney, Winona, MN (US)

(72) Inventor: Samuel J. Forney, Winona, MN (US)

(21) Appl. No.: 19/385,358

(22) Filed: Nov. 11, 2025

Related U.S. Application Data
(60) Provisional application No. 63/178,809, filed on Apr. 23, 2021.

(51) Int. Cl. A61M 21/02; A41D 1/04; A61M 21/00

(52) U.S. Cl. CPC A61M 21/02; A41D 1/04; A41D 2400/32; A61M 2021/0022

(57) ABSTRACT

A therapeutic vest includes an interior base portion and an exterior weighted portion. The interior base portion includes an interior back panel, an interior right panel, and an interior left panel. The exterior weighted portion includes a back panel, a right panel and a left panel. The exterior weighted portion is fixedly attached to the interior base portion. The exterior weighted portion includes a plurality of enclosures. Each of the plurality of enclosures is configured to contain a media of a predetermined volume and mass.

### BACKGROUND

Weighted therapeutic vests can be worn by a user to ease anxiety through deep pressure stimulation, activating the parasympathetic nervous system. Weighted therapeutic vests can provide proprioceptive input using deep pressure which sends signals to the brain which, as a result, helps a person feel calm and increase focus. For adults and children with autism, for example, a therapeutic vest can be a sensory instrument to help reduce anxiety, increase focus, concentration, and self-regulation.

### BRIEF DESCRIPTION OF THE DRAWINGS

FIG. 1A is front view of a therapeutic vest, in accordance with one embodiment;

FIG. 1B is a back view of the therapeutic vest, in accordance with the embodiment of FIG. 1A;

FIG. 2 is an expanded view of a front portion of a therapeutic vest, in accordance with the embodiment of FIG. 1A;

FIG. 3 is an expanded view a back portion of the therapeutic vest, in accordance with the embodiment of FIG. 1B;

FIG. 4 is another view of the therapeutic vest, in accordance with the embodiment of FIGS. 1A-1B;

FIG. 5A is an enlarged view of the side fasteners in a tightened position of the therapeutic vest, in accordance with the embodiment of FIG. 4;

FIG. 5B is an enlarged view of the side fasteners in a loosened position, in accordance with the embodiment of FIG. 4;

FIGS. 6A and 6B are front and side views of a therapeutic vest, in accordance with another embodiment;

FIG. 7A is front view of a therapeutic vest, in accordance with one embodiment; and

FIG. 7B is a back view of the therapeutic vest, in accordance with the embodiment of FIG. 7A.

### DETAILED DESCRIPTION

In the following detailed description, reference is made to the accompanying drawings which form a part hereof, and in which is shown by way of illustration specific examples in which the invention may be practiced. In this regard, directional terminology, such as “top”, “bottom”, “front”, “back”, “leading”, “trailing”, etc., is used with reference to the orientation of the Figures(s) being described. Because components of examples can be positioned in a number of different orientations, the directional terminology is used for purposes of illustration and is in no way limiting. It is to be understood that other examples may be utilized and structural or logical changes may be made without departing from the scope of the present invention. The following detailed description, therefore, is not to be taken in a limiting sense, and the scope of the present invention is defined by the claims. It is to be understood that features of the various examples described herein may be combined with each other, unless specifically noted otherwise.

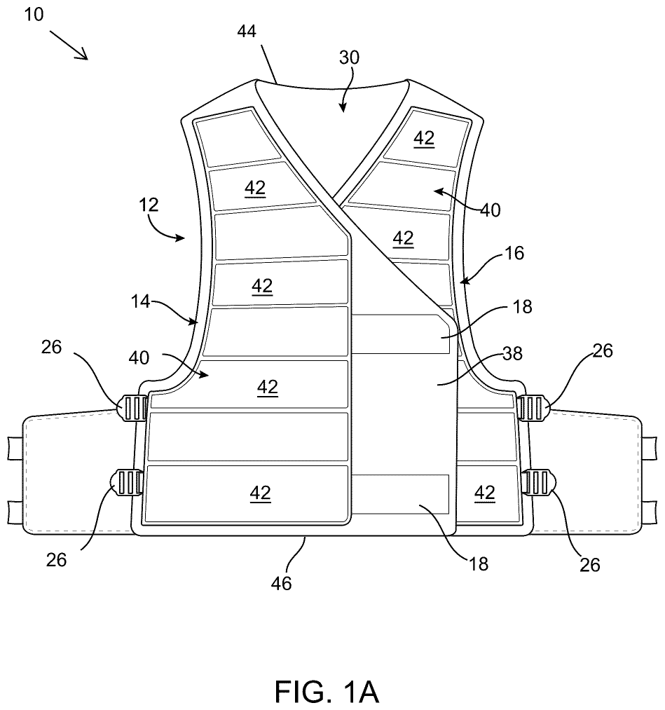

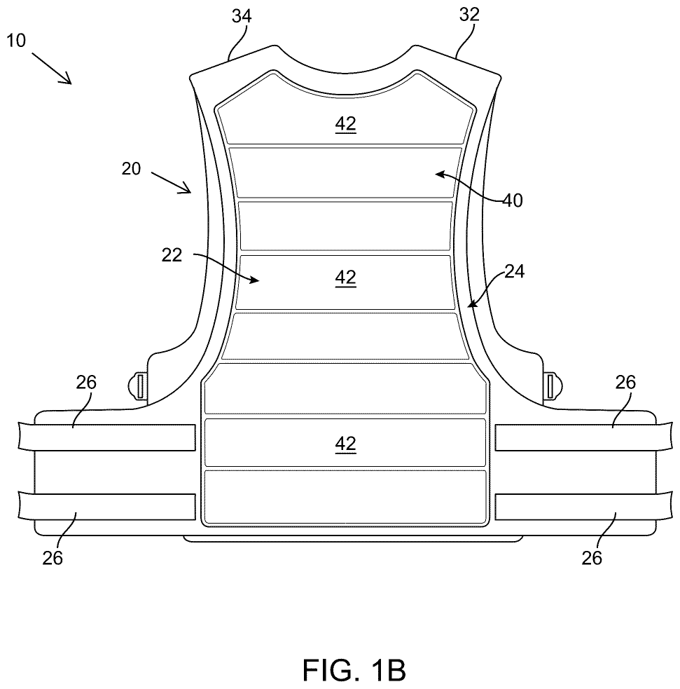

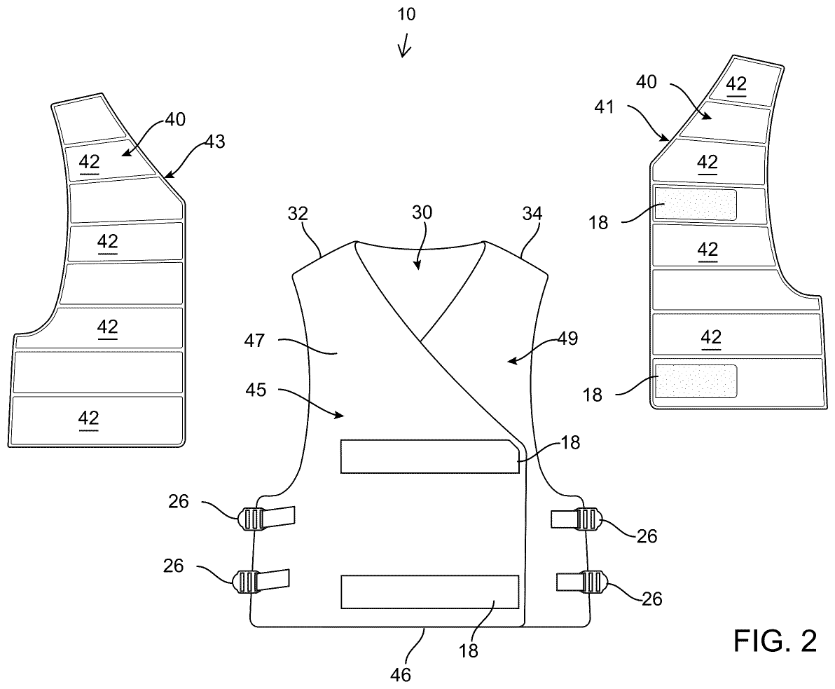

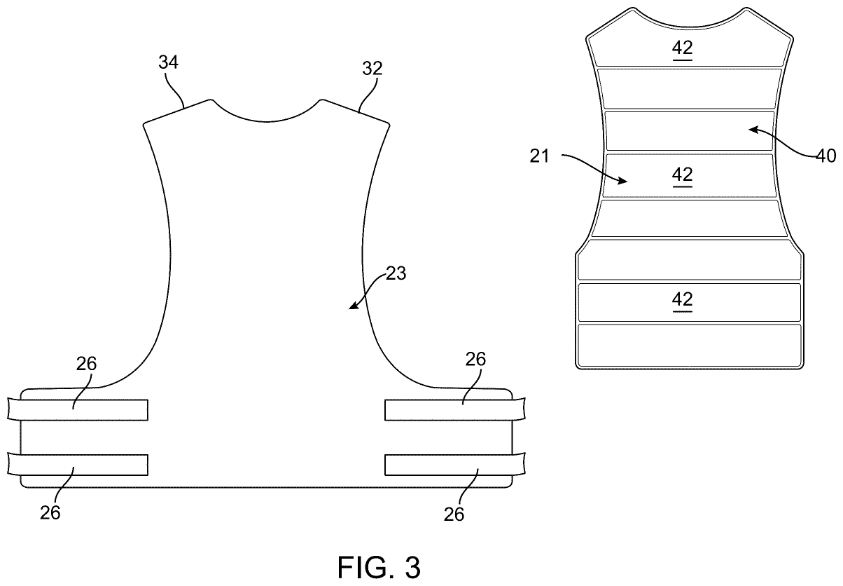

FIGS. 1A and 1B illustrate front and back views of a therapeutic vest 10, in accordance with one embodiment. As illustrated in FIG. 1A, a front portion 12 of the therapeutic vest 10 includes a right section 14 and a left section 16. The right and left sections 14, 16 can be releasably coupled together and uncoupled from one another with a fastening means 18 in order to allow a user to put on and take off the therapeutic vest 10, as described further below. As illustrated in FIG. 1B, a back portion 20 of the therapeutic vest 10 extends between opposing right and left sides 22, 24, to substantially cover a back of a user or wearer. With continued reference to FIGS. 1A and 1B, the right and left sections 14, 16 of the front portion 12 may be coupled to the back portion 20 along the right and left sides 22, 24. The therapeutic vest 10 may also include tightening means 26 extending between the front and back portions 12, 14, such as adjustable straps, for example. The right side and left sections 14, 16 of the front portion 12 may be coupled with the back portion 20 to form an interior cavity 30 for the wearer or user. With additional reference to FIGS. 2 and 3, the therapeutic vest 10 includes an exterior weighted portion 40 formed by a series of enclosures 42 extending between right and left edges of the right and left sections 14, 16 of the front portion 12 and right and left sides 22, 24 of the back portion 20. The series of enclosures 42 can be aligned across and around the therapeutic vest 10, extending in parallel between the neck opening 44 and the bottom edge 46.

The tightening means 26 can be used for adjusting the interior cavity 30 of the therapeutic vest 10 to conform to the size of the torso of the user, as well as allowing for varying degrees of tightness of the therapeutic vest 10 on the user. The tightening means 26 may be disposed along each of the left and right sides 22, 24 and can draw the right and left sections 14, 16 of the front portion 12, at varying degrees, closer to or further apart from the back portion 20. In one embodiment, each of the right and left sides 22, 24 of the therapeutic vest 10 include corresponding tightening means 26. In one embodiment, more than one tightening means 26 is included on each of the opposing left and right sides 22, 24. In one embodiment, the tightening means 26 is comprised of a strap and a buckle, with the strap threaded through the buckle and the strap adjustably pullable or pushable through openings of the buckle. In another embodiment, the tightening means 26 is comprised of mating hook and loop portions, for example, Velcro®. In another embodiment, the tightening means 26 is comprised of ties or strings connected or looped to connectors on portions of the therapeutic vest 10 for adjusting the tightness of the therapeutic vest 10. Other forms of adjustable tightening means 26 are also acceptable.

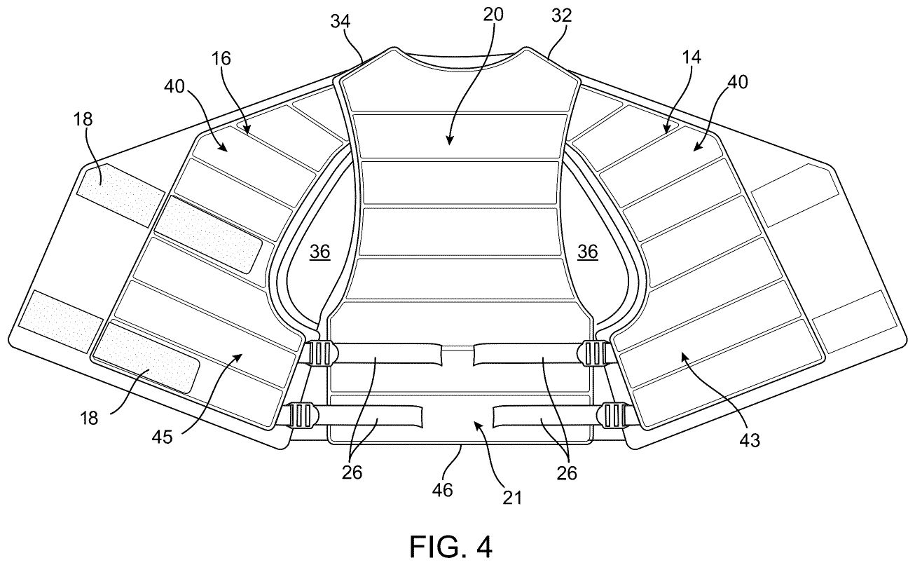

As illustrated, a top portion of the therapeutic vest 10 defines a neck opening 44. The neck opening 44 is open to the interior cavity 30. The neck opening 44 is positioned between right and left shoulder areas 32, 34 formed at the top portion of the therapeutic vest 10. A bottom edge 46 is defined generally opposite of the shoulder areas 32, 34 and is configured to be disposed along a wearer's waist. With additional reference to FIG. 4, the therapeutic vest 10 includes arm openings 36. The arm openings 36 extend along, or between the front and back portions 12, 20 of the therapeutic vest 10. The arm openings 36 are open to the interior cavity 30. In one embodiment, the arm openings 36 are oversized to allow a loose fit around the wearer's arms. The arm openings 36 can extend from the shoulder areas 32, 34 generally to the tightening means 26. In one embodiment, the arm openings 36 extend a majority of distance between the shoulder areas 32, 34 and the bottom edge 46.

With particular reference to FIGS. 1A and 2, the therapeutic vest 10 also includes a front fastening means 18 for releasably coupling the right and left sections 14, 16 of the front portion 12 together. In one embodiment, the fastening means 18 can be included on a flap 38 on one or both of the right or left sections 14, 16. In one embodiment, the front fastening means 18 is configured to securely couple the right and left sections 14, 16 such that they will not inadvertently become uncoupled. In one embodiment, the fastening means 18 can be comprised of at least two sets of mating hook and loop sections extending in parallel along the flap 38 below the neck opening 44 of the right and left sections 14, 16, respectively. In another embodiment, the fastening means 18 can be comprised of at least two sets of mateable hook and loop sections spaced between the bottom edge 46 and the neck opening 44 along joining edges of the right and left sections 14, 16. In one embodiment, the fastening means 18 is comprised of at least two or more of mating hook and loop sections, although other numbers of sets of mating hook and loop sections are also acceptable. Fastening means 18 can be any one or combination of buttons and buttonholes or loops, snaps, zippers, clips or other suitable means of releasably and securely coupling the right and left sections 14, 16 of the front portion 12 together.

The weighted portion(s) 40 of the therapeutic vest 10 may be comprised by a series of enclosures 42, or compartments, extending between right and left edges of the right and left sections 14, 16 of the front portion 12 and right and left edges of the back portion 20. In one embodiment, the weighted portions 40 do not extend onto the flap 38 and are absent from the flap 38. In one embodiment, the weighted portions 40 do not extend onto left and right side areas beneath the arm openings 36 that are adjustable between tightened and loosened positions of the tightening means 26.

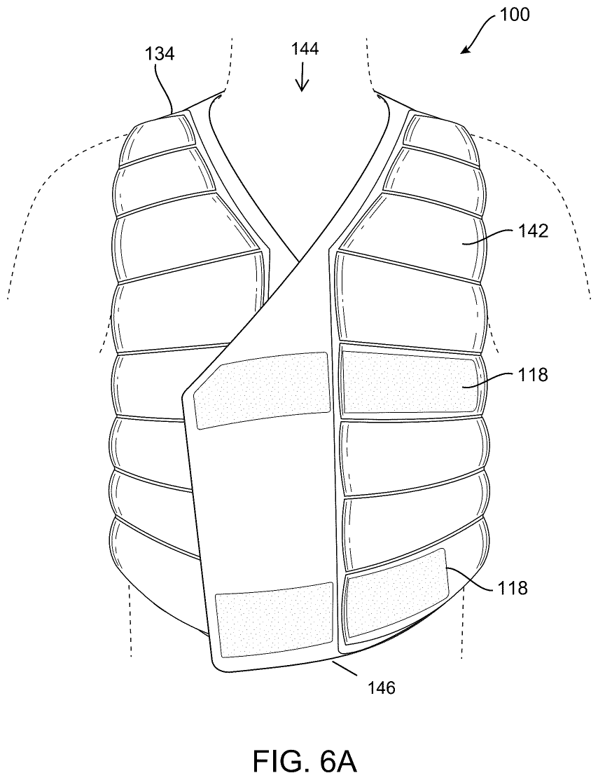

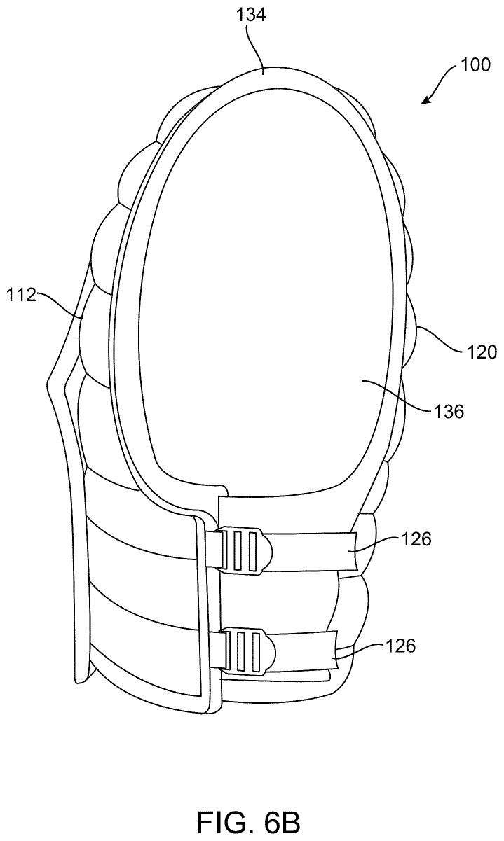

With additional reference to FIGS. 6A and 6B, the therapeutic vest 100 is similar to the therapeutic vest 10, and like elements are similarly numbered. Each of the series of enclosures 42, 142 extend generally horizontally across a body of the wearer. In one embodiment, the series of enclosures 42, 142 extend in parallel between the neck opening 44, 144 and the bottom edge 46, 146 and are aligned in a row-like formation along the front portion 12, 112 and back portion 20, 120 of the therapeutic vest 10, 100. In one embodiment, each of the enclosures 42, 142 along the back portion 20, 120 have a width (e.g., in a direction between the neck opening and the bottom edge) that is less than a length (e.g., between opposing right and left sides). In one embodiment, a length of the plurality increases from the top portion to the bottom edge 46, 146 of the therapeutic vest 10, 100 on at least the front portion 12, 112. In one embodiment, a width of the series of enclosures 42 is equivalent from the shoulder areas 34, 134 to the lower edge 46, 146 of the therapeutic vest 10, 100. In one embodiment, the width of the enclosures 42 is approximately 2″ (two inches), as measured in a vertical or top to bottom direction on a wearer, and the length of the enclosures 42, 142 vary to correspond to the therapeutic vest 10, 100. Other enclosure widths and variations may also be acceptable.

With further reference in particular to the expanded views of FIGS. 2 and 3, FIG. 2 is an expanded front view of the therapeutic vest 10 and FIG. 3 is an assembled front view of the therapeutic vest 10, in accordance with the embodiment of FIGS. 1A and 1B. The front portion 12 of the therapeutic vest 10 includes a right section weighted portion 41 and a left section weighted portion 43, or right and left panel exterior weighted panels 41, 43. The front portion 12 of the vest further includes a front base 45 formed of interior right and left panels 47, 49. The right and left weighted portions 41, 43 correspond to in shape and size, and are configured to be attached to, the right and left interior panels 47, 49, respectively. In one embodiment, the back portion 20 is assembled with the front base portion 45 on the interior and the right and left front weighted panels 47, 49 on the exterior when worn.

The back portion 20 of the therapeutic vest 10 includes a back section weighted portion 21, or back exterior weighted panel 21. The back portion 20 of the therapeutic vest 10 further includes a back base 23, or a back panel 23. In one embodiment, the back portion 20 is assembled with back base portion 23 on the interior and the back weighted portion 21 on the exterior when worn.

The weighted portions 21, 43, 45 are coupled with the corresponding base portions 23, 47, 49 with stitching or other suitable means. In addition, as illustrated in FIG. 4, the base portion 45 of the back portion 20 is attached or coupled to the right and left panels 47, 49 of the front portion 12 along the top of the shoulder areas 32, 34 adjacent to the neck opening 44 via stitching, for example.

The interior base portions 23, 47, 49 is comprised of a soft-to-the-touch fabric that is suitably strong to support the attachment of the exterior weighted portions 21, 43, 45. In one embodiment, the fabric has a tight weave for increased strength and durability. In one embodiment, the base portion(s) 23, 43, 45 includes more than one layer of material that are sewn together along perimeter edges. In one embodiment, the base portion provides a padded layer that is comfortable for the wearer. In one embodiment, the base portion 23, 47, 49 is comprised of at least one layer of a fabric that is durable and soft, such as flannel, for example. In one embodiment, the durable and soft fabric is a Japanese flannel. In one embodiment, the interior base is comprised of at least one layer of the fabric, at least one layer of a stabilizer (e.g., interfacing), and at least one layer of padding. In one embodiment, the padding is comprised of cotton batting. In one embodiment, the interior base portions 23, 47, 49 is comprised of an inside layer of fabric, at least one layer of batting disposed along the inside layer of fabric, a layer of the stabilizer disposed along an opposing side of the at least one layer of batting, and an exterior layer of fabric disposed along the layer of stabilizer opposite the batting. The interior and exterior layers can be comprised of the same or different types of fabric. In some embodiments, additional layers of the fabric, the batting and/or the stabilizer can be included. In one embodiment, the layers of the fabric, the batting, and the stabilizer are sewn together along perimeter edges with the layers of fabric fully enclosing the layers of the batting and the stabilizer. In one embodiment, at least a portion of the fastening means 18 is included on the front base portion. In one embodiment, one mateable portion of the fastening means 18 is included on the right or left base portion and another mateable portion is includes on the opposite section (left or right) of the weighted portion. In one embodiment, the front and back sections of the base portions 23, 47, 49 include side adjusting means.

The weighted portions 21, 43, 45 may be comprised of at least two layers of material coupled together to form the series of enclosures 42 containing the weighted material between the layers of material. In one embodiment, each of the at least two layers of material include a layer or interfacing material and a layer of fabric that is the same or similar to that the base portion 23, 47, 49 is comprised of. In one embodiment, each of the layers of interfacing material is adjacent to the weighted material and each of the layers of fabric are disposed on the opposing side of the interfacing material from the weighted material.

Additionally referencing FIGS. 6A and 6B, the weighted material is flowable within each of the respective enclosures 42, 142. In one embodiment, the weighted material is formed of a mixed media of various densities and masses of media. In one embodiment, a total weight of the weighted material of the front portion 12, 112 is equivalent to a total weight of the weighted material of the back portion 20, 120. In one embodiment, the weighted material has a total weight of three to ten pounds distributed within the series of front and back enclosures 42, 142. In one embodiment, the weighted material is approximately ten percent of the body weight of a wearer of the therapeutic vest 10, 100.

The weighted portions 40, 140 including the weighted material within the enclosures 42, 142 are configured to be conformable, in particular, conformable to the wearer's body. The enclosures 42, 142 or compartments, are closed around an entirety of a perimeter of each of the plurality of enclosures 42, 142 in order to fully contain the weighted material (not shown). In one embodiment, the enclosures 42, 142 are sewn shut, or otherwise sealed, in order to contain the weighted material. The weighted material is contained within the enclosures 42, 142 and is not configured to be adjusted or removed by a user or wearer.

The weighted material is disposed within each of the enclosures 42 ,142 such that the weighted material is flowable within each of the enclosures 42, 142. In other words, the volume of the weighted material is less than the available volume within each of the enclosures 42, 142 in order that the weighted material is movable within each of the enclosures 42, 142. In one embodiment each of the enclosures 42 is formed by a least one row of stitching forming a perimeter of the respective enclosure 42, 142. In one embodiment, at least two rows of stitching are used along the perimeter of the respective enclosures 42, 142. In one embodiment, the perimeter stitching can be a shared stitching between two adjacent enclosures 42, 142. In one embodiment, the top shoulder areas 32, 34, 134 of the wearer there do not include the enclosures 42, 142.

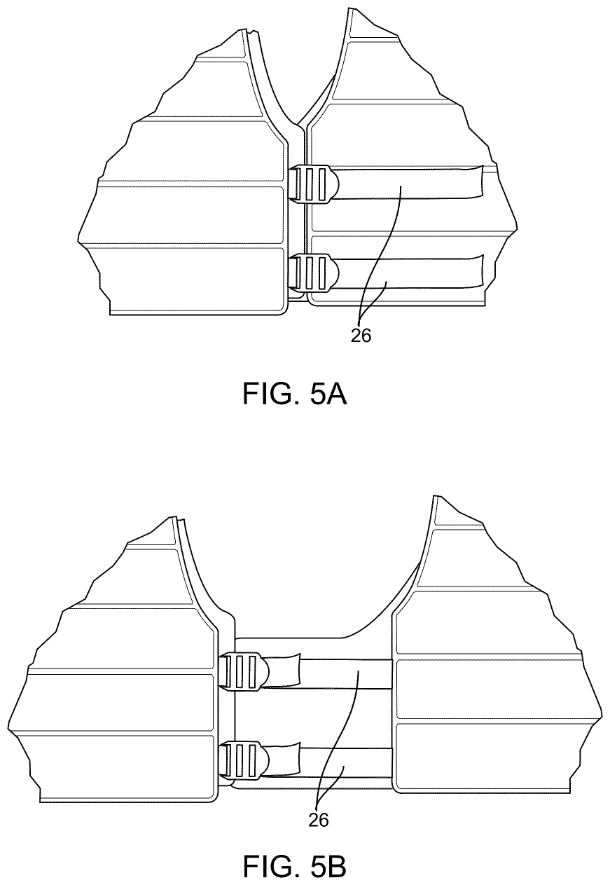

As illustrated in FIGS. 5A and 5B the tightening means 26 may be included along the sides of the therapeutic vest 10 below the arm openings 36. In one embodiment, each of the tightening means 26 can be independently adjustable. In one embodiment, each of the respective left and right sides 22, 24 of the therapeutic vest 10 can include one or more tightening means 26. In one embodiment, the respective left and right side tightening means 26 includes two tightening means 26 comprised of mateably adjustable tightening means 26. In one embodiment, the tightening means 26 are the means by which the left and right front portions 12 are attached to the back portion 20. In another embodiment, the left and right front portions 12 are also attached to the back portion 20 with stitching or other means. FIG. 5A illustrates the tightening means 26 fully, or nearly fully, in a tightened configuration. In the tightened configuration the weighted portion of the back section is drawn closer to the respectively adjacent weighted portion of the front section than in the loosened configuration illustrated in FIG. 5B. In FIG. 5B, the weighted portion 40 of the back portion 20 is extended away from the weighted portion 40 of the respectively adjacent weighted portion 40 of the front section 12. In one embodiment, the mateable portions of the tightening means 26 can be fully disengaged. In one embodiment, the tightening means 26 can be mateable hook and loop portions, adjustable strap and buckle, or other suitable means of adjustable tightening and loosening.

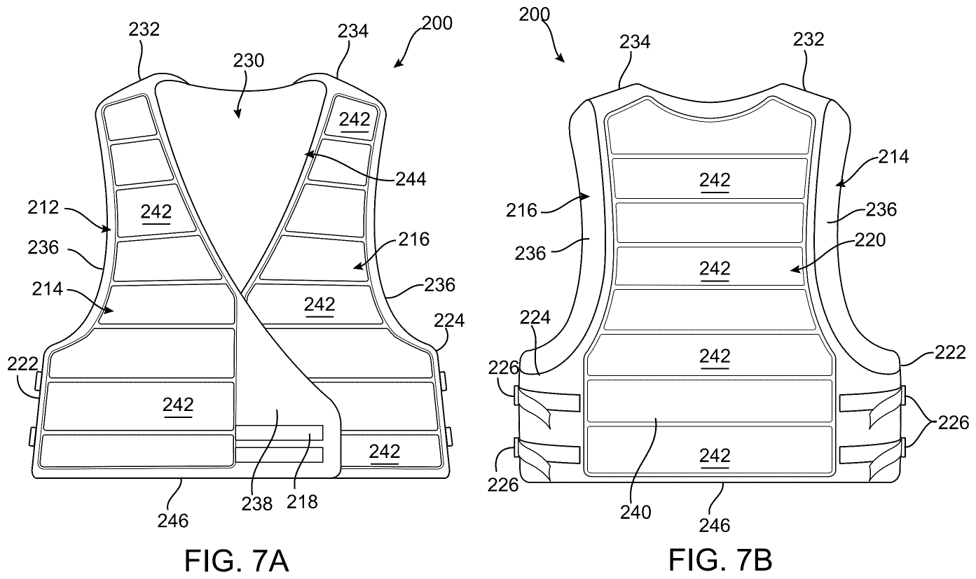

FIGS. 7A and 7B illustrate another embodiment of a therapeutic vest 200 of the present disclosure. The therapeutic vest 200 is similar to the therapeutic vests 10, 100 and like elements are similarly numbered. The therapeutic vest 200 of FIGS. 7A and 7B includes fastening means 218 as a series of fastening means 218 each spaced apart from the adjacent fastening means 218 along a flap 238 of the front sections between a neck opening 244 and a bottom edge 246. In this embodiment, the flap 238 extends such as to leave a larger neck opening 244 between a top or shoulder and the bottom edge 246 of the therapeutic vest 200 than in the embodiments of therapeutic vests 10, 100.

One aspect of the disclosure relates to a therapeutic vest including an interior base portion including an interior back panel, an interior right panel, and an interior left panel. The therapeutic vest includes an exterior weighted portion having a back panel, a right panel and a left panel. The exterior weighted portion is fixedly attached to the interior base portion. The exterior weighted portion includes a plurality of enclosures. Each of the plurality of enclosures is configured to contain a media of a predetermined volume and mass.

One aspect of the disclosure relates to a therapeutic vest, wherein the exterior weighted portion is configured to be fixedly coupled to the interior base portion along perimeter edges of the interior base portion.

One aspect of the disclosure relates to a therapeutic vest, wherein the interior base portion is comprised of at least one layer of filling material, and a layer of fabric disposed on opposing faces of the filling material.

One aspect of the disclosure relates to a therapeutic vest, wherein the interior base portion is further comprised of a layer of strengthening material.

One aspect of the disclosure relates to a therapeutic vest, wherein the exterior weighted portion includes a first layer and a second layer, wherein the first layer and the second layer are coupled to form the plurality of enclosures.

One aspect of the disclosure relates to a therapeutic vest, wherein the enclosures of the weighted portion are configured to extend across a width of a wearer's body.

One aspect of the disclosure relates to a therapeutic vest, wherein the enclosures are sealed.

One aspect of the disclosure relates to a therapeutic vest, wherein the media is non-removable.

One aspect of the disclosure relates to a therapeutic vest, wherein the media mass occupies a portion of a volume of each of the plurality of enclosures.

One aspect of the disclosure relates to a therapeutic vest, wherein the media is flowable within the each of the plurality of enclosures.

One aspect of the disclosure relates to a therapeutic vest, wherein a mass of the media of the back panel of the exterior weighted portion is equivalent to a combined mass of the media of the right and left panels of the exterior weighted portion.

One aspect of the disclosure relates to a therapeutic vest including a back portion, a front portion, a front fastening means, and opposing left and right side adjustable means. The back portion includes an interior back portion and an exterior weighted back portion. The exterior weighted back portion contains a flowable media. The interior back portion and the exterior weighted back portion are correspondingly shaped and sized. The front portion includes a right side having a right front interior panel and a right front weighted portion attached to the right front interior panel, and a left side having a left front interior panel and a left front weighted portion attached to the left front interior panel. The right and left front weighted portions each contain a flowable media. The right front interior panel and the right front weighted portion are correspondingly shaped and sized. The left front interior panel and the left front weighted portion are correspondingly shaped and sized.

One aspect of the disclosure relates to a therapeutic vest, wherein the flowable media is sealed within a plurality of enclosures.

One aspect of the disclosure relates to a therapeutic vest, wherein the plurality of enclosures is a series of horizontally extending rows, wherein the first of the plurality of enclosures is positioned along a shoulder area and the last of the plurality of enclosures is positioned along a waist area.

One aspect of the disclosure relates to a therapeutic vest, wherein the front fastening means is configured to releasably secure the right and left sides of the front portion.

One aspect of the disclosure relates to a therapeutic vest, wherein the front fastening means is comprised of two rows of hook and loop sections.

One aspect of the disclosure relates to a therapeutic vest, wherein the interior back portion, the right front interior panel and the left front interior panel are each comprised of at least one layer of filling material, and a layer of fabric disposed on opposing faces of the filling material.

One aspect of the disclosure relates to a therapeutic vest, wherein the plurality of enclosures is formed between a first layer and an opposing second layer of the weighted portions.

One aspect of the disclosure relates to a therapeutic vest, wherein the flowable media occupies a portion of a volume of each of the plurality of enclosures.

One aspect of the disclosure relates to a therapeutic vest including a base portion and a weighted portion. The base portion has an upper area defining a neck opening, a right side and a left side defining arm openings, and a lower edge. The weighted portion is fixedly attached to the base portion. The weighted portion includes at least two layers of fabric. The two layers are attached together to form a series of enclosures. Each enclosure is configured to contain media. The media is moveable within each of the enclosures.

As used in the specification and the appended claims, the singular forms “a”, “an”, and “the” include plural referents unless the context clearly dictates otherwise. Ranges may be expressed herein as from “about” or “approximately” one particular value and/or to “about”or “approximately”another particular value. When such a range is expressed, another embodiment includes from the one particular value and/or to the other particular value. Similarly, when values are expressed as approximations, by use of the antecedent “about”, it will be understood that the particular value forms another embodiment.

It should be noted that embodiments and features described in the context of one of the aspects of the present invention also apply to the other aspects of the invention.

1. A therapeutic vest comprised of:
an interior base portion comprised of an interior back panel, an interior right panel, and an interior left panel; and
an exterior weighted portion comprised of a back panel, a right panel and a left panel,
wherein the exterior weighted portion is fixedly attached to the interior base portion, and wherein the exterior weighted portion comprised of a plurality of enclosures, wherein each of the plurality of enclosures is configured to contain a media of a predetermined volume and mass.

2. The therapeutic vest of claim 1, wherein the exterior weighted portion is configured to be fixedly coupled to the interior base portion along perimeter edges of the interior base portion.

3. The therapeutic vest of claim 1, wherein the interior base portion is comprised of at least one layer of filling material, and a layer of fabric disposed on opposing faces of the filling material.

4. The therapeutic vest of claim 2, wherein the interior base portion is further comprised of a layer of strengthening material.

5. The therapeutic vest of claim 1, wherein the exterior weighted portion includes a first layer and a second layer, wherein the first layer and the second layer are coupled to form the plurality of enclosures.

6. The therapeutic vest of claim 1, wherein the enclosures of the weighted portion are configured to extend across a width of a wearer's body.

7. The therapeutic vest of claim 1, wherein the enclosures are sealed.

8. The therapeutic vest of claim 1, wherein the media is non-removable.

9. The therapeutic vest of claim 1, wherein the media mass occupies a portion of a volume of each of the plurality of enclosures.

10. The therapeutic vest of claim 1, wherein the media is flowable within the each of the plurality of enclosures.

11. The therapeutic vest of claim 1, wherein a mass of the media of the back panel of the exterior weighted portion is equivalent to a combined mass of the media of the right and left panels of the exterior weighted portion.

12. A therapeutic vest comprised of:
a back portion comprised of an interior back portion and an exterior weighted back portion, wherein the exterior weighted back portion contains a flowable media, and wherein the interior back portion and the exterior weighted back portion are correspondingly shaped and sized;
a front portion comprised of a right side having a right front interior panel and a right front weighted portion attached to the right front interior panel, and a left side having a left front interior panel and a left front weighted portion attached to the left front interior panel, wherein the right and left front weighted portions each contain a flowable media, and wherein the right front interior panel and the right front weighted portion are
correspondingly shaped and sized, and wherein the left front interior panel and the left front weighted portion are correspondingly shaped and sized;
a front fastening means; and
opposing left and right side adjustable means.

13. The therapeutic vest of claim 12, wherein the flowable media is sealed within a plurality of enclosures.

14. The therapeutic vest of claim 12, wherein the plurality of enclosures is a series of horizontally extending rows, wherein the first of the plurality of enclosures is positioned along a shoulder area and the last of the plurality of enclosures is positioned along a waist area.

15. The therapeutic vest of claim 12, wherein the front fastening means is configured to releasably secure the right and left sides of the front portion.

16. The therapeutic vest of claim 15, wherein the front fastening means is comprised of at least two rows of hook and loop sections.

17. The therapeutic vest of claim 12, wherein the interior back portion, the right front interior panel and the left front interior panel are each comprised of at least one layer of filling material, and a layer of fabric disposed on opposing faces of the filling material.

18. The therapeutic vest of claim 13, wherein the plurality of enclosures is formed between a first layer and an opposing second layer of the weighted portions.

19. The therapeutic vest of claim 13, wherein the flowable media occupies a portion of a volume of each of the plurality of enclosures.

20. A therapeutic vest comprised of:
a base portion having an upper area defining a neck opening, a right side and a left side defining arm openings, and a lower edge; and
a weighted portion fixedly attached to the base portion, the weighted portion comprising at least two layers of fabric, the two layers attached together to form a series of enclosures, each enclosure configured to contain media, the media moveable within each of the enclosures.
